(19) United States
(12) Patent Application Publication
(10) Pub. No.: US 2016/0002240 A1
(43) Pub. Date: Jan. 7, 2016

(54) NUCLEOSIDE ANALOG SALTS WITH IMPROVED SOLUBILITY AND METHODS OF FORMING SAME

(71) Applicant: THE BOARD OF TRUSTEES OF THE UNIVERSITY OF ALABAMA, Tuscaloosa, AL (US)

(72) Inventor: Robin D. Rogers, Tuscaloosa, AL (US)

(21) Appl. No.: 14/770,655

(22) PCT Filed: Mar. 17, 2014
(86) PCT No.: PCT/US14/30234

§ 371 Date: Aug. 26, 2015

Related U.S. Application Data
(60) Provisional application No. 61/793,613, filed on Mar. 15, 2013.

(51) Int. Cl. C07D 473/18; C07C 309/17; C07F 9/54; C07C 215/40; C07C 211/63

(52) U.S. Cl. CPC C07D 473/18; C07C 215/40; C07C 211/63; C07F 9/5407; C07C 309/17

(57) ABSTRACT

Disclosed are salts of nucleoside analogs and methods of forming the salts. An anion of a nucleoside analog is paired with a permanent counter cation to form a salt that has decreased melting point and increased aqueous solubility compared to the nucleoside compound prior to the salt formation. Also a cation of a nucleoside analog is paired with a permanent counter anion to form a salt that has decreased melting point and increased aqueous solubility compared to the nucleoside compound prior to the salt formation. The nucleoside analog in some embodiments has therapeutic activity such as antiviral. The permanent counter cation or anion in some embodiments has bioactivity such as antibacterial or being a vitamin.

### CROSS-REFERENCE TO RELATED APPLICATIONS

This application claims the benefit of priority to U.S. Provisional Application No. 61/793,613, filed Mar. 15, 2013, which is hereby incorporated herein by reference in its entirety.

### FIELD

The subject matter disclosed herein generally relates to compositions and to methods of preparing nucleoside analog salts with improved aqueous solubility.

### BACKGROUND

Acyclovir is the most commonly used antiviral drug, it is a nucleoside analog of the guanosine family. It is widely used as tablets, topical cream, intravenous injection, and ophthalmic ointment. Acyclovir is particularly known as an anti-Herpes drug, being used in the treatment of Herpes genitalis, Herpes simplex, Herpes zoster, and Epstein-Barr. Although widely used, it suffers from limited solubility in water, resulting in a low oral bioavailability (of only 10-20%) and the necessity for intravenous administration in high doses. Therefore, research in this area is currently focused on improving the bioavailability of acyclovir.

There are several strategies to improve the oral absorption and plasma level of the poorly soluble acyclovir. Current technologies rely mainly on the formation of prodrugs. The most widely used approach is the formation of a corresponding ester or other prodrug-type compound (Colla et al., J Med Chem 1983, 26(4):602-604; Beauchamp et al., Antiviral Chem Chemother 1992, 3:157-164). For example, valacyclovir, the most common ester prodrug of acyclovir (obtained by esterification of acyclovir with the amino acid valine), has an oral bioavailability of about 55% (MacDougall et al., J Antimicrob Chemother 2004, 53:899-901), which is much higher than that of acyclovir. The disadvantage of using this approach is the multistep syntheses of these prodrugs.

Other strategies to improve the poor water solubility of antiviral acyclovir deal with formulations and compositions of acyclovir (Arnalet et al. J Pharm Sci 2008, 97(12):5061-5073; USPGP 2008-0107749), nitrate salts of acyclovir (WO 2001/00116), and arylsulfonic acid salts (WO 2001/051485). There are also a few examples of acyclovir salts in the literature where acyclovir anion is paired with metal cations (e.g., acyclovir sodium salt (U.S. Pat. No. 6,040,445), acyclovir lithium salt (EP 135312)). However, new forms of acyclovir, as well as similar nucleosides, with improved water solubility are still needed. The methods and compositions disclosed herein address these and other needs.

### SUMMARY

In accordance with the purposes of the disclosed materials, compounds, compositions, and methods, as embodied and broadly described herein, the disclosed subject matter, in one aspect, relates to salts of nucleoside analogs, such as acyclovir, and methods for preparing and using such salts.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying Figures, which are incorporated in and constitute a part of this specification, illustrate several aspects described below.

FIG. 1 is a comparison of the infrared spectra of acyclovir, [Cho][Acy] (1), [N_{4,4,4,1}][Acy] (2), [P_{4,4,4,4}][Acy] (4), [N_{4,4,4,4}][Acy] (5) and potassium acyclovir.

FIG. 2 is a comparison of the infrared spectra of acyclovir, [H₂Acy]Cl (6), [H₂Acy][Doc] (7), and silver docusate.

FIG. 3 depicts the thermal stability of acyclovir, [Cho][Acy] (1), [N_{4,4,4,1}][Acy] (2), [N_{1,1,1,16}][Acy] (3), [P_{4,4,4,4}][Acy] (4), [N_{4,4,4,4}][Acy] (5) and [H₂Acy]Cl (6).

FIG. 4 shows the powder x-ray diffractograms of acyclovir, [P_{4,4,4,4}][Acy] (4), [H₂Acy]Cl (6), and [H₂Acy][Doc] (7).

FIG. 5 shows images of ChoAcy solution in water (a) after mixing for 24 h at room temperature; (b) mixture from (a) after standing for 16 days at room temperature; (c) aliquot taken from (a) after centrifugation and standing for 15 days at room temperature.

FIG. 6 shows images of [Acy][Doc] in water: (a) 0.2 g/1.5 mL mixture after 24 h of stirring at room temperature; (b) mixture from (a) after 48 h of standing at room temperature.

FIG. 7 depicts the differential scanning calorimetry (DSC) profile of [Cho][Acy] (1).

FIG. 8 depicts the DSC profile of [N_{4,4,4,1}][Acy] (2).

FIG. 9 depicts the DSC profile of [P_{4,4,4,4}][Acy] (4).

FIG. 10 depicts the DSC profile of [N_{4,4,4,4}][Acy] (5).

FIG. 11 depicts the DSC profile of [H₂Acy][Doc] (7).

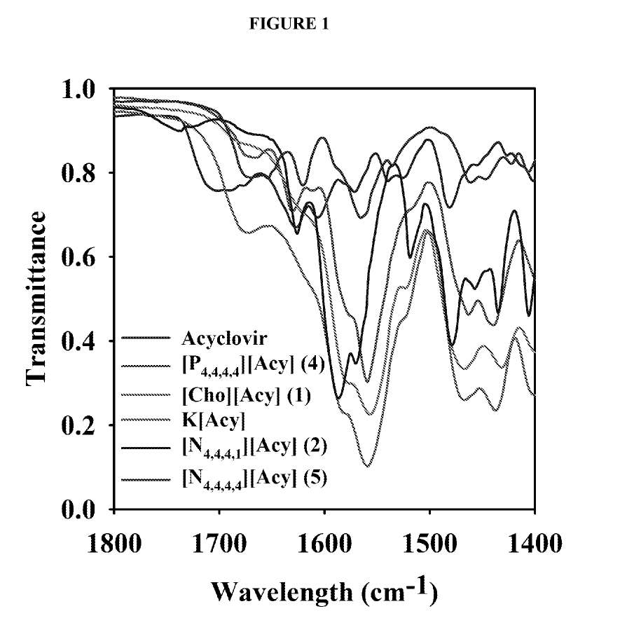

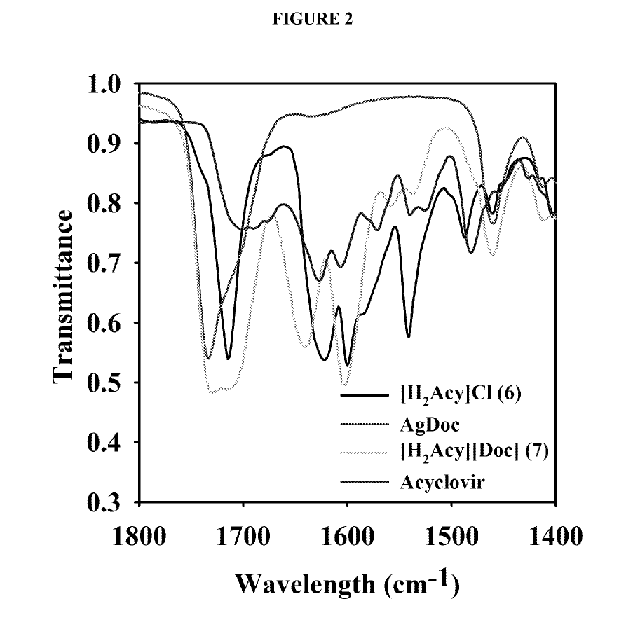

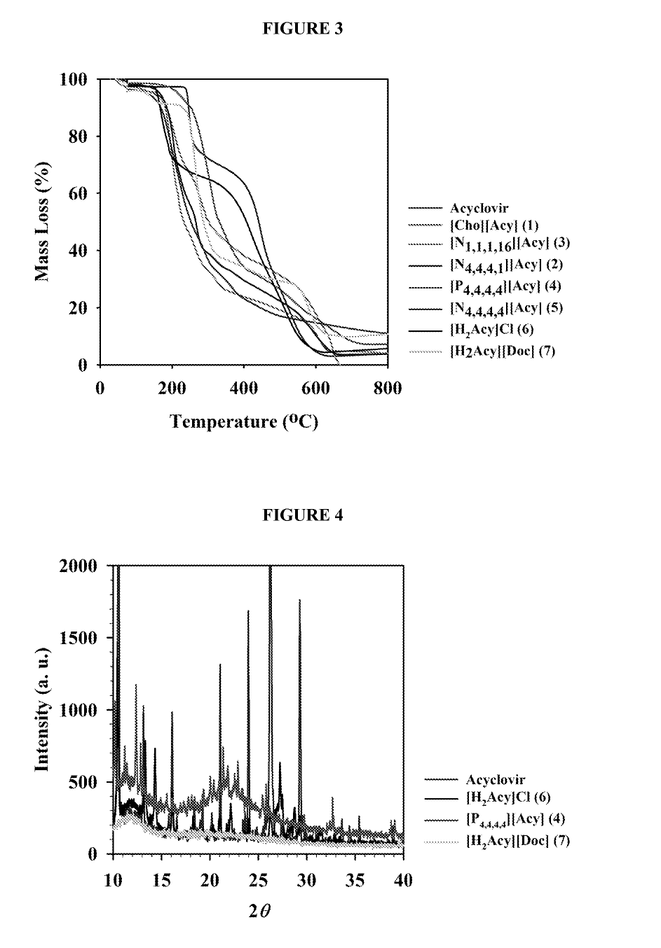

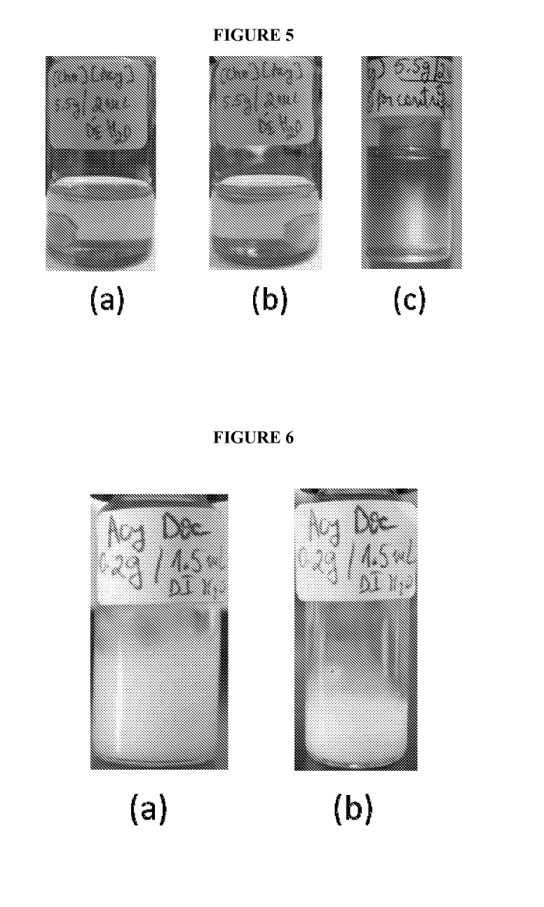

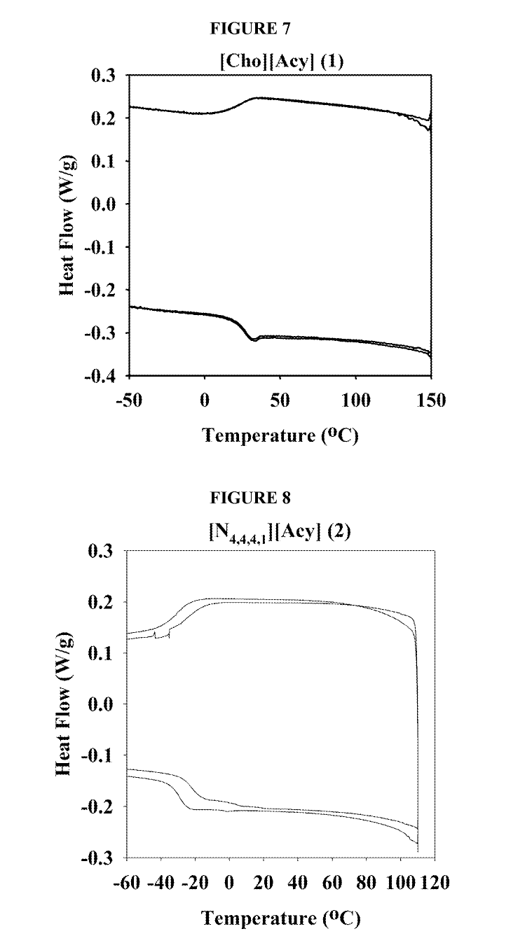

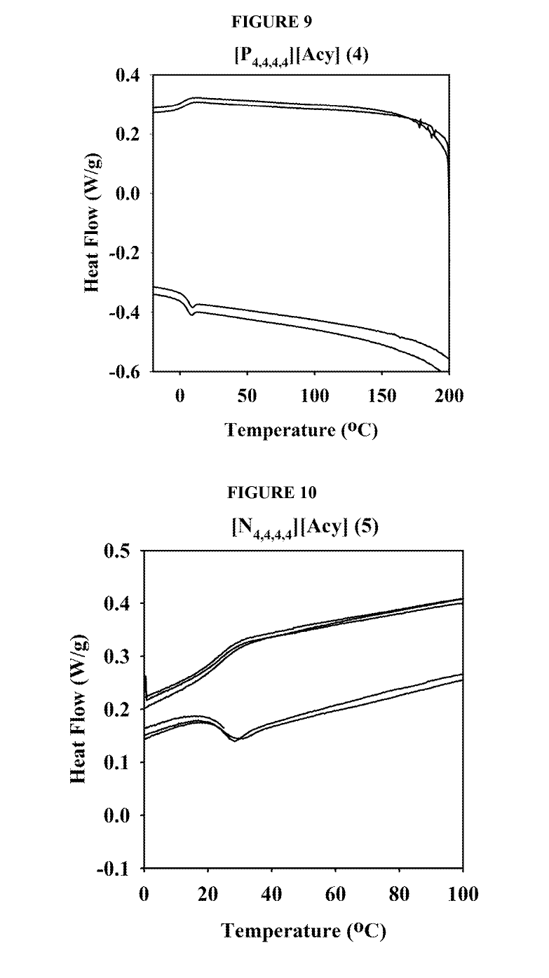

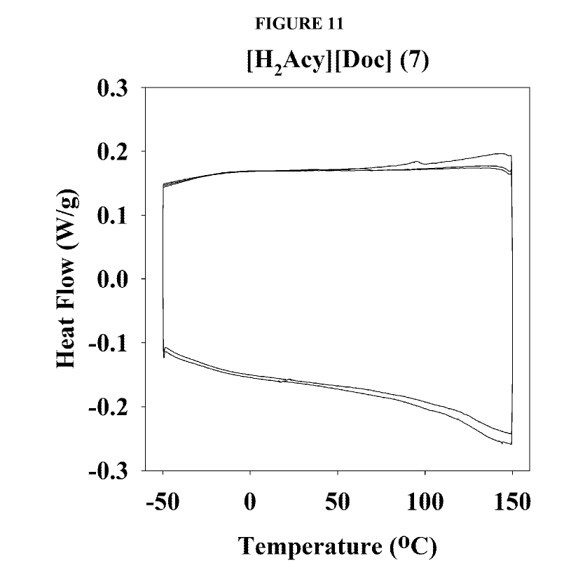

### DETAILED DESCRIPTION

Disclosed herein is an approach that uses ionic liquids as a tool to improve the aqueous solubility of acyclovir and similar nucleoside-analog antivirals. Thus, provided herein are salts of nucleoside analogs and methods of forming the salts. The nucleoside analog salts described herein contain, in one aspect, an anion of a nucleoside analog and a permanent cation as the counter ion to improve the solubility of the nucleoside analog. Alternatively, the nucleoside analog salts described herein contain a cation of a nucleoside analog and a permanent anion as the counter ion to improve the solubility of the nucleoside analog. Some of the nucleoside analogs possess therapeutic activity, such as antiviral activity. Optional additional bioactivity can be introduced to the salt through permanent counter cations or permanent counter anions, as the case may be, that are themselves bioactive, e.g., having antimicrobial activities or being a vitamin.

The disclosed salts in some aspects can be ionic liquids and can be used in that form. However, ionic liquids need not actually be prepared and used. Thus, in other aspects, a salt where cations and anions, which together are capable of forming an ionic liquid, are dissolved in a solution. While not wishing to be bound by theory, it is believed that as a result of the ionic liquid forming propensity of the particular cations and anions used, the salts described herein can achieve improved solubility and physical properties. In addition, the combination of two or more active chemicals in a single salt can introduce secondary biological function.

The compounds, compositions, and methods described herein can be understood more readily by reference to the following detailed description of specific aspects of the disclosed subject matter and the Examples and Figures included therein.

Before the present compounds, salts, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an ionic liquid” includes mixtures of two or more such ionic liquids, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. Unless otherwise stated, “about” means within 5% of the stated value, for example within 1% of the stated value.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### CHEMICAL DEFINITIONS

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., Zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, deesterification, hydrolysis, etc.

The term “anion” is a type of ion and is included within the meaning of the term “ion.” An “anion” is any molecule, portion of a molecule (e.g., Zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., Zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkyl alcohol” is used in another, it is not meant to imply that the term “alkyl” does not also refer to specific terms such as “alkyl alcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

The term “bioactive property” is any local or systemic biological, physiological, or therapeutic effect in a biological system. For example, the bioactive property can be the control of pesticidal, herbicidal, nutritional, antimicrobial, fungicidal, an algaecidal, insecticidal, miticidal, molluscicidal, nematicidal, rodenticidal, virucidal action, penetration enhancer, etc. Many examples of these and other bioactive properties are disclosed herein.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed compounds, compositions, and methods, examples of which are illustrated in the accompanying Examples and Figures.

### Materials and Compositions

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), Sigma (St. Louis, Mo.), Pfizer (New York, N.Y.), GlaxoSmithKline (Raleigh, N.C.), Merck (Whitehouse Station, N.J.), Johnson & Johnson (New Brunswick, N.J.), Aventis (Bridgewater, N.J.), AstraZeneca (Wilmington, Del.), Novartis (Basel, Switzerland), Wyeth (Madison, N.J.), Bristol-Myers-Squibb (New York, N.Y.), Roche (Basel, Switzerland), Lilly (Indianapolis, Ind.), Abbott (Abbott Park, Ill.), Schering Plough (Kenilworth, N.J.), Akzo Nobel Chemicals Inc (Chicago, Ill.), Degussa Corporation (Parsippany, N.J.), Monsanto Chemical Company (St. Louis, Mo.), Dow Agrosciences LLC (Indianapolis, Ind.), DuPont (Wilmington, Del.), BASF Corporation (Florham Park, N.J.), Syngenta US (Wilmington, Del.), FMC Corporation (Philadelphia, Pa.), Valent U.S.A. Corporation (Walnut Creek, Calif.), Applied Biochemists Inc (Germantown, Wis.), Rohm and Haas Company (Philadelphia, Pa.), Bayer CropScience (Research Triangle Park, N.C.), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials can be obtained from commercial sources.

In one aspect, disclosed herein are ionic liquids. The term “ionic liquid” is used herein to refer to salts (i.e., compositions comprising cations and anions) that are liquid at a temperature of at or below about 150° C. That is, at one or more temperature ranges or points at or below about 150° C. the disclosed ionic liquid compositions are liquid; although, it is understood that they can be solids at other temperature ranges or points. See e.g., Wasserscheid and Keim, Angew Chem Int Ed Engl, 2000, 39:3772; and Wasserscheid, “Ionic Liquids in Synthesis,” 1^{st} Ed., Wiley-VCH, 2002. Further, exemplary properties of ionic liquids are high ionic range, non-volatility, non-flammability, high thermal stability, wide temperature for liquid phase, highly solvability, and non-coordinating. For a review of ionic liquids see, for example, Welton, Chem Rev 1999, 99:2071-2083; and Carlin et al., Advances in Nonaqueous Chemistry, Mamantov et al. Eds., VCH Publishing, New York, 1994.

The term “liquid” describes the ionic liquid compositions that are generally in amorphous, non-crystalline, or semi-crystalline state. For example, while some structured association and packing of cations and anions can occur at the atomic level, the ionic liquid compositions can have minor amounts of such ordered structures and are therefore not crystalline solids. The compositions can be fluid and free-flowing liquids or amorphous solids such as glasses or waxes at temperatures at or below about 150° C. In some examples described herein, the ionic liquid compositions are liquid at the temperature at which the composition is applied (i.e., ambient temperature).

Further, the disclosed ionic liquid compositions are materials composed of at least two different ions, each of which can independently and simultaneously introduce a specific characteristic to the composition not easily obtainable with traditional dissolution and formulation techniques. Thus, by providing different ions and ion combinations, one can change the characteristics or properties of the disclosed ionic liquid compositions in a way not seen by simply preparing various crystalline salt forms.

Examples of characteristics that can be controlled in the disclosed compositions include, but are not limited to, melting point, solubility control, stability, and biological activity or function. It is this multi-nature/functionality of the disclosed ionic liquid compositions which allows one to fine-tune or design in very specific desired material properties.

It is further understood that the disclosed ionic liquid compositions can include solvent molecules (e.g., water); however, these solvent molecules are not required to be present in order to form the ionic liquids. That is, the disclosed ionic liquid compositions can contain, at some point during preparation and application, no or minimal amounts of solvent molecules that are free and not bound or associated with the ions present in the ionic liquid composition. The disclosed ionic liquid compositions can, after preparation, be further diluted with solvent molecules (e.g., water) to form a solution suitable for application. Thus, the disclosed ionic liquid compositions can be converted into liquid hydrates, solvates, or solutions. In regard to the solutions, they need not be referred to as an original from a diluted ionic liquid. The solutions disclosed herein can arise by separately dissolving the cations and anions in a solvent. It is understood that solutions formed by diluting ionic liquids or by separately dissolving the cations and anions that could form an ionic liquid possess enhanced chemical properties that are unique to ionic liquid-derived solutions.

The specific physical properties (e.g., melting point, viscosity, density, water solubility, etc.) of ionic liquids are determined by the choice of cation and anion, as is disclosed more fully herein. As an example, the melting point for an ionic liquid can be changed by making structural modifications to the ions or by combining different ions. Similarly, the particular chemical properties (e.g., toxicity, bioactivity, etc.), can be selected by changing the constituent ions of the ionic liquid.

Since many ionic liquids are known for their non-volatility, thermal stability, and ranges of temperatures over which they are liquids, the deficiencies of nucleoside analogs such as poor aqueous solubility can be addressed through the formation of ionic liquids or solutions of ions that are capable of forming ionic liquids, rather than covalent modification of the nucleoside analog itself. The salts disclosed herein are comprised of at least one kind of anion and at least one kind of cation. In these salts, either the at least one kind of anion or the at least one kind of cation can possess a bioactive property. For example, the anion or cation possessing the bioactive property can be antiviral, antimicrobial such as antibacterial, or nutritional such as a vitamin. Additionally, the anion or cation possessing the bioactive property can be an antimicrobial active, an anti-inflammatory active, or an anti-tumor active, or the like, including any combination thereof, as is disclosed herein. It is contemplated that the disclosed ionic liquid compositions can comprise one kind of cation with more than one kind of anion (e.g., 2, 3, 4, 5, 6, 7, 8, 9, 10 or more different anions). Likewise, it is contemplated that the disclosed ionic liquid compositions can comprise one kind of anion with more than one kind of cation (e.g., 2, 3, 4, 5, 6, 7, 8, 9, 10, or more different kinds of cations). Further, the disclosed ionic liquids can comprise more than one kind of anion (e.g., 2, 3, 4, 5, 6, 7, 8, 9, 10, or more different kinds of anions) with more than one kind of cation (e.g., 2, 3, 4, 5, 6, 7, 8, 9, 10 or more different kinds of cations). Specific examples include, but are not limited to, one kind of cation with 1, 2, 3, 4, 5, 6, 7, 8, 9, 10, or more kind of anions, 2 kinds of cations with 1, 2, 3, 4, 5, 6, 7, 8, 9, 10, or more kinds of anions, 3 kinds of cations with 1, 2, 3, 4, 5, 6, 7, 8, 9, 10, or more kinds of anions, 4 kinds of cations with 1, 2, 3, 4, 5, 6, 7, 8, 9, 10, or more kinds of anions, 5 kinds of cations with 1, 2, 3, 4, 5, 6, 7, 8, 9, 10, or more kinds of anions, 6 kinds of cations with 1, 2, 3, 4, 5, 6, 7, 8, 9, 10, or more kinds of anions, 7 kinds of cations with 1, 2, 3, 4, 5, 6, 7, 8, 9, 10, or more kinds of anions, 8 kinds of cations with 1, 2, 3, 4, 5, 6, 7, 8, 9, 10, or more kinds of anions, 9 kinds of cations with 1, 2, 3, 4, 5, 6, 7, 8, 9, 10, or more kinds of anions, 10 kinds of cations with 1, 2, 3, 4, 5, 6, 7, 8, 9, 10, or more kinds of anions, or more than 10 kinds of cations with 1, 2, 3, 4, 5, 6, 7, 8, 9, 10, or more kinds of anions.

Other specific examples include, but are not limited to, one kind of anion with 1, 2, 3, 4, 5, 6, 7, 8, 9, 10, or more kinds of cations, 2 kinds of anions with 1, 2, 3, 4, 5, 6, 7, 8, 9, 10, or more kinds of cations, 3 kinds of anions with 1, 2, 3, 4, 5, 6, 7, 8, 9, 10, or more kinds of cations, 4 kinds of anions with 1, 2, 3, 4, 5, 6, 7, 8, 9, 10, or more kinds of cations, 5 kinds of anions with 1, 2, 3, 4, 5, 6, 7, 8, 9, 10, or more kinds of cations, 6 kinds of anions with 1, 2, 3, 4, 5, 6, 7, 8, 9, 10, or more kinds of cations, 7 kinds of anions with 1, 2, 3, 4, 5, 6, 7, 8, 9, 10, or more kinds of cations, 8 kinds of anions with 1, 2, 3, 4, 5, 6, 7, 8, 9, 10, or more kinds of cations, 9 kinds of anions with 1, 2, 3, 4, 5, 6, 7, 8, 9, 10, or more kinds of cations, 10 kinds of anions with 1, 2, 3, 4, 5, 6, 7, 8, 9, 10, or more kinds of cations, or more than 10 kinds of anions with 1, 2, 3, 4, 5, 6, 7, 8, 9, 10, or more kinds of cations.

In addition to the cations and anions, the salts disclosed herein can be used to form compositions that also contain nonionic liquid species, such as solvents, preservatives, dyes, colorants, thickeners, surfactants, viscosity modifiers, mixtures and combinations thereof and the like. The amount of such nonionic liquid species can range from less than about 99, 90, 80, 70, 60, 50, 40, 30, 20, or 10 wt. % based on the total weight of the composition. In some examples described herein, the amount of such nonionic liquid species is low (e.g., less than about 10, 9, 8, 7, 6, 5, 4, 3, 2, or 1 wt. % based on the total weight of the composition). In some examples described herein, the disclosed ionic liquid salts are neat; that is, the only materials present in the disclosed ionic liquids are the cations and anions that make up the ionic liquids. It is understood, however, that with neat salts, some additional materials or impurities can sometimes be present, albeit at low to trace amounts (e.g., less than about 10, 9, 8, 7, 6, 5, 4, 3, 2, or 1 wt. % based on the total weight of the composition).

The disclosed compositions, when in ionic liquid form, are liquid at some temperature range or point at or below about 150° C. For example, the disclosed ionic liquids can be a liquid at or below about 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0, −1, −2, −3, −4, −5, −6, −7, −8, −9, −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, −45, −46, −47, −48, −49, −50, −51, −52, −53, −54, −55, −56, −57, −58, −59, −60, −61, −62, −63, −64, −65, −66, −67, −68, −69, −70, −71, −72, −73, −74, −75, −76, −77, −78, −79, −80, −81, −82, −83, −84, −85, −86, −87, −88, −89, −90, −91, −92, −93, −94, −95, −96, −97, −98, −99, or −100° C., where any of the stated values can form an upper or lower endpoint of a range. In further examples, the disclosed ionic liquids can be liquid at any point from about −30° C. to about 150° C., from about −20° C. to about 140° C., from about −10° C. to about 130° C., from about 0° C. to about 120° C., from about 10° C. to about 110° C., from about 20° C. to about 100° C., from about 30° C. to about 90° C., from about 40° C. to about 80° C., from about 50° C. to about 70° C., from about −30° C. to about 50° C., from about −30° C. to about 90° C., from about −30° C. to about 110° C., from about −30° C. to about 130° C., from about −30° C. to about 150° C., from about 30° C. to about 90° C., from about 30° C. to about 110° C., from about 30° C. to about 130° C., from about 30° C. to about 150° C., from about 0° C. to about 100° C., from about 0° C. to about 70° C., from about 0° to about 50° C., and the like.

Further, in some examples the disclosed ionic liquid compositions can be liquid over a wide range of temperatures, not just a narrow range of, for example, 1-2 degrees. For example, the disclosed ionic liquid compositions can be liquids over a range of at least about 4, 5, 6, 7, 8, 9, 10, or more degrees. In other examples, the disclosed ionic liquid compositions can be liquid over at least about an 11, 12, 13, 14, 15, 16, 17, 18, 19, 20, or more degree temperature range. Such temperature ranges can begin and/or end at any of the temperature points disclosed in the preceding paragraph.

As described above, it is understood that the disclosed ionic liquid compositions can be formulated in an extended or controlled release vehicle, for example, by encapsulating the compositions in microspheres or microcapsules using methods known in the art. Still further, the disclosed compositions can themselves be solvents for other solutes. These and other formulations of the disclosed compositions are disclosed elsewhere herein.

The disclosed salts can be substantially free of water in some examples (e.g., immediately after preparation of the salts and before any further application of the salts). By substantially free is meant that water is present at less than about 10, 9, 8, 7, 6, 5, 4, 3, 2, 1, 0.5, 0.25, or 0.1 wt. %, based on the total weight of the salts.

The salts disclosed herein have increased solubility compared to the nucleoside compound prior to the salt formation. For example, the aqueous solubility of the salt is at least 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, or 5000 times that of the nucleoside compound prior to salt formation, where any of the stated values can form an upper or lower endpoint of a range. In embodiments when the salt is a liquid at ambient temperature, the liquid salt is considered miscible with water.

The disclosed compositions can be prepared by methods described herein. Generally, the particular cation(s) and anion(s) used to prepare an ionic liquid are selected as described herein to form the salts as disclosed herein. The resulting ionic liquid salt can be then used in the ionic liquid form or diluted in a suitable solvent as described herein. Additionally, the method for the preparation of the disclosed salts can include the metathesis reaction between two salt species: one salt containing the anion (e.g., anion of nucleoside) and the other salt containing the permanent counter cation are combined, resulting in the salts as disclosed herein. Again, such an ionic liquid can be used as is or diluted in an appropriate solvent. Still further, the disclosed compositions can be prepared by mixing the precursor of the anion with a solution of the cation, wherein the cations and anions are capable of forming the salt, albeit under different nonsolvating conditions.

Nucleoside Analog Anions

As described above, the at least one anion is anion of a nucleoside analog. An example of a nucleoside analog acyclovir is shown below.

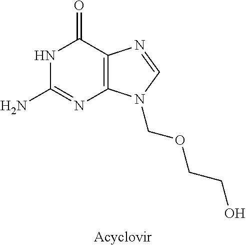 [SMILES: Nc1nc2c(ncn2COCCO)c(=O)[nH]1]

When the aromatic NH group (position 1 of the purine) is deprotonated, as shown in scheme 1 below, it forms an anion of acyclovir. The negative charge at position 1 is further delocalized to position 2, position 3, as well as to the carbonyl group as indicated in scheme 1, providing stability to the anion.

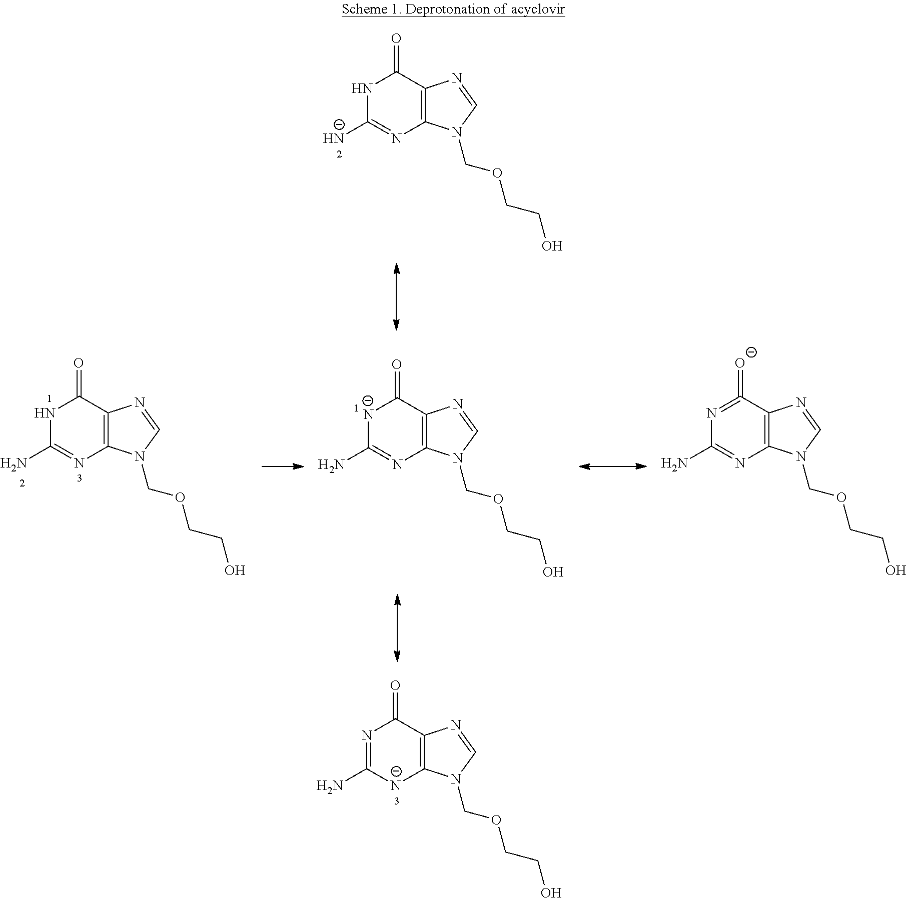

Besides guanosine-based nucleoside analogs, such as acyclovir, other nucleoside analogs that can be deprotonated to form anions can be similarly converted to the salt forms described herein to increase their solubility. These nucleoside analogs are referred to as nucleoside compounds, nucleobase-derived compounds, or simply nucleosides throughout the present disclosure. For example, nucleoside analogs, such as those disclosed in Scheme 2, can have a purine or pyrimidine moiety that can be deprotonated to form an anion, the negative charge of which can similarly be delocalized to provide stability to the anions. Many drugs are nucleoside analogs, such as the compounds listed in Scheme 3. Similar to acyclovir, the aqueous solubility of these drugs can be significantly improved by forming a salt with a permanent counter cation, as disclosed herein. When the drug compound is deprotonated to form an anion, the negative charge can delocalize to provide stability to the anion. Although only example compounds from the nucleoside analog drug family are presented in Scheme 3, prodrugs of these drugs can similarly be deprotonated, forming delocalized anions that form salts with permanent cation counter ions with improved solubility.

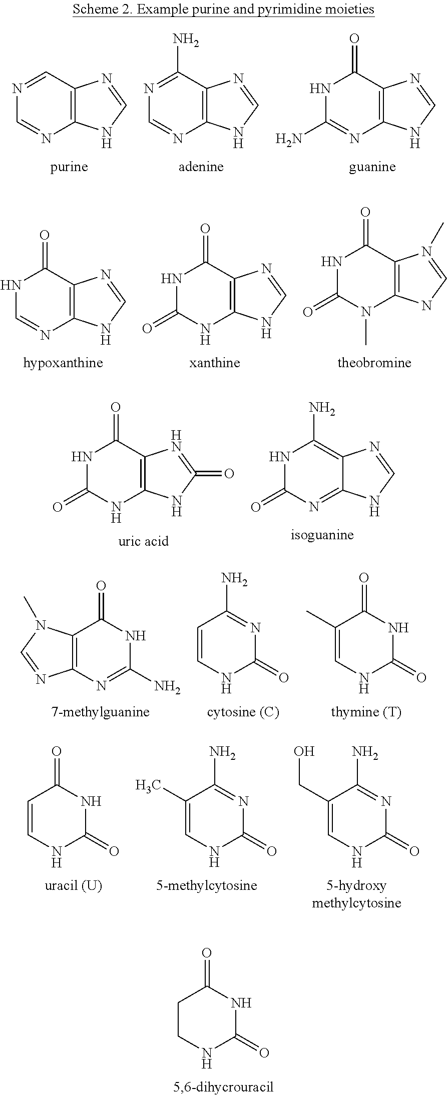

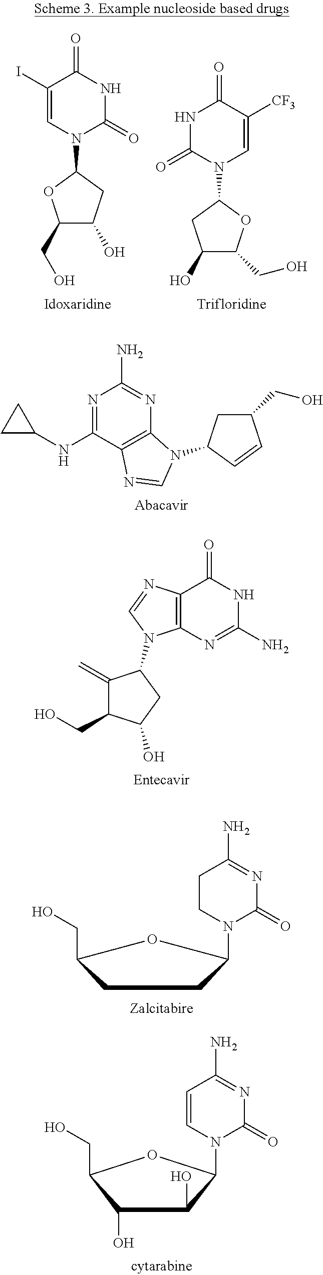

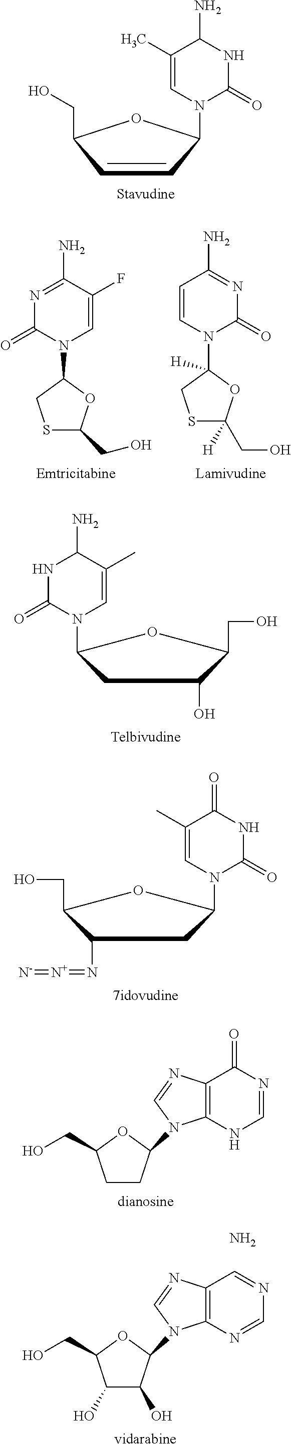

Any of these nucleosides or nucleoside based drugs can be combined with any of the permanent cations disclosed herein in accordance with the disclosed subject matters. Some additional nucleoside analogs than can be suitable anions of the disclosed salts include: A-5021, famciclovir, penciclovir, and ganciclovir.

Permanent Cations

As described above, the at least one cation can be a permanent cation. The term “permanent cation” is used to include any non-protic cation, particularly containing saturated quaternary nitrogen (e.g., N,N,N,N-tetraalkylammonium and N,N-dialkylpyrrolidinium), saturated quaternary nitrogen (e.g., N-substituted pyridinium, picolinium, or N,N-disubstituted imidazolium), and phosphorous and sulfur-containing analogues (e.g., tetraalkylphosphonium and trialkylsulfonium).

In some examples, the permanent cation used herein can comprise an ammonium cation of the structure ⁺NR¹R²R³R⁴, wherein R¹, R², R³, and R⁴ are each independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted heteroaryl, or substituted or unsubstituted carbonyl.

In some examples, the permanent cation used herein can comprise a phosphonium cation of the structure ⁺PR¹R²R³R⁴, wherein R¹, R², R³, and R⁴ are each independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted heteroaryl, or substituted or unsubstituted carbonyl.

In some examples, the permanent cation used herein can comprise a sulfonium cation of the structure ⁺SR¹R²R³, wherein R¹, R², and R³ are each independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted heteroaryl, or substituted or unsubstituted carbonyl.

In some examples, the permanent cation used herein can comprise a N,N-disubstituted pyrrolidinium or a N,N-disubstituted imidazolium cation of the following structure,

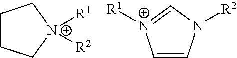

wherein R¹ and R² are each independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted heteroaryl, or substituted or unsubstituted carbonyl. In one embodiment, the permanent cation is Ethylmethyl-imidazolium.

In some examples, the permanent cation used herein can comprise a N-substituted pyridinium or a N-substituted picolinium of the following structure,

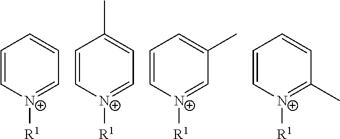

wherein R¹ is selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted heteroaryl, or substituted or unsubstituted carbonyl.

Particular examples of permanent cations that can be present in the disclosed salts as bioactive cations are compounds that contain nitrogen or phosphorus atoms. Nitrogen atom-containing groups can be neutral or can be converted to positively-charged quaternary ammonium species, for example, through alkylation or protonation of the nitrogen atom. Thus, compounds that possess a quaternary nitrogen atom (known as quaternary ammonium compounds (QACs)) are typically cations. According to the methods and compositions disclosed herein, any compound that contains a quaternary nitrogen atom or a nitrogen atom that can be converted into a quaternary nitrogen atom can be a suitable cation for the disclosed compositions. In some examples, the cation is not a protonated amine or a metal.

QACs can have numerous biological properties that may be desired in the disclosed compositions. For example, many QACs are known to have antibacterial properties. The antibacterial properties of QACs were first observed toward the end of the 19^{th} century among the carbonium dyestuffs, such as auramin, methyl violet, and malachite green. These types of compounds are effective chiefly against Gram-positive organisms. Jacobs and Heidelberger first discovered the antibacterial effects of QACs in 1915, when studying the antibacterial activity of substituted hexamethylene-tetrammonium salts (Jacobs and Heidelberger, Proc Nat Acad Sci USA, 1915, 1:226; Jacobs and Heidelberger, J Biol Chem, 1915, 20:659; Jacobs and Heidelberger, J Exptl Med, 1916, 23:569, which are incorporated by reference herein in their entireties for their teachings of various cations).

Browning et al. found great, and somewhat less selective, bactericidal power among quaternary derivatives of pyridine, quinoline, and phenazine (Browning et al., Proc Roy Soc London, 1922, 93B:329; Browning et al., Proc Roy Soc London, 1926, 100B:293 which are incorporated by reference herein in their entireties for their teachings of various cations). Hartman and Kagi observed antibacterial activity in QACs of acylated alkylene diamines (Hartman and Kagi, Z Angew Chem, 1928, 4:127, which is incorporated by reference herein in its entirety for its teachings of various cations).

In 1935, Domagk synthesized long-chain QACs, including benzalkonium chloride, and characterized their antibacterial activities (Domagk, Deut Med Wochenschr, 1935, 61:829 which is incorporated by reference herein in its entirety for its teachings of various cations). He showed that these salts are effective against a wide variety of bacterial strains. This study of the use of QACs as germicides was greatly stimulated.

Many scientists have focused their attention on water soluble QACs because they exhibit a range of properties: they are surfactants, they destroy bacteria and fungi, they serve as a catalyst in phase-transfer catalysis, and they exhibit anti-electrostatic and anticorrosive properties. Water soluble QACs can exert antibacterial action against both Gram-positive and Gram-negative bacteria as well as against some pathogen species of fungi and protozoa. These multifunctional salts have also been used in wood preservation (Oertel, Holztechnologie, 1965, 6:243; Butcher et al., For Prod J, 1977, 27:19; Butcher et al., J For Sci, 1978, 8:403, which are incorporated by reference herein in their entireties for their teachings of various cations).

Many examples of compounds having nitrogen atoms, which exist as quaternary ammonium species or can be converted into quaternary ammonium species, are disclosed herein. Some specific QACs suitable for use herein include aliphatic heteroaryls (i.e., a compound that comprises at least one aliphatic moiety bonded to a heteroaryl moiety), aliphatic benzylalkyl ammonium cation (i.e., a cation that comprises an aliphatic moiety bonded to the nitrogen atom of a benzylalkyl amine moiety), dialiphatic dialkyl ammonium cations (i.e., a compound that comprises two aliphatic moieties and two alkyl moieties bonded to a nitrogen atom), a tetraalkyl ammonium cation, or other quaternary ammonium cations.

The permanent cation can also include substituted or unsubstituted benztriazoliums, substituted or unsubstituted benzimidazoliums, substituted or unsubstituted benzothiazoliums, substituted or unsubstituted pyridiniums, substituted or unsubstituted pyridaziniums, substituted or unsubstituted pyrimidiniums, substituted or unsubstituted pyraziniums, substituted or unsubstituted imidazoliums, substituted or unsubstituted pyrazoliums, substituted or unsubstituted oxazoliums, substituted or unsubstituted 1,2,3-triazoliums, substituted or unsubstituted 1,2,4-triazoliums, substituted or unsubstituted thiazoliums, substituted or unsubstituted piperidiniums, substituted or unsubstituted pyrrolidiniums, substituted or unsubstituted quinoliums, and substituted or unsubstituted isoquinoliums.

Examples of suitable bioactive permanent cations are listed in Table 1 below. Any of the cations disclosed herein, and preferably those in Table 1, can be combined with acyclovir to form a salt according to the disclosed subject matter herein.

TABLE 1
Examples of suitable bioactive cations

| Permanent Cation | | Bioactivity |
|---|---|---|
| choline | 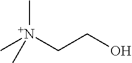 | Vitamin |
| P₄₄₄₄ | 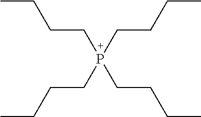 | Antibacterial |
| N₄₄₄₄ | 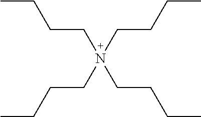 | Antibacterial |
| N₄₄₄₁ | 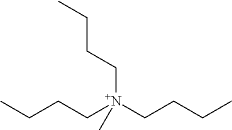 | Antibacterial |
| N₁₁₁₁₆ | 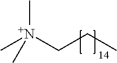 | Antibacterial |

Nucleoside Analog Cations

Also disclosed herein are compositions where the at least one cation is a cation of a nucleoside analog. For example, the nucleoside analog acyclovir, shown above, can be protonated (e.g., at position 2 as shown below).

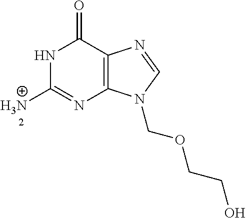 [SMILES: [NH3+]c1nc2c(ncn2COCCO)c(=O)[nH]1]

The positive charge can be delocalized to other carbon or nitrogen atoms in the compound, providing stability to the cation. In addition to protonation with an acid, converting the base portion of a nucleoside analog into a cation can also be accomplished by alkylating with, e.g., an alkyl halide. All of the nucleoside analogs disclosed above as examples of suitable anions, can be similarly converted to their cationic forms. Thus, the nucleoside analogs disclosed above are expressly referenced herein in their cationic forms.

Permanent Anions

The cationic nucleoside analogs disclosed herein can be combined with one or more permanent anions. Suitable anions include a halide (fluoride, chloride, bromide, or iodide) or C₁-C₆ carboxylate. Less preferred anions include hypochlorite, chlorite, perchlorate, cyanide (CN⁻), thiocyanate (SCN⁻), cyanate (OCN⁻), fulminate (CNO⁻), azide (N₃⁻), tetrafluoroborate (BF₄), and hexafluorophosphate (PF₆) anions.

Carboxylate anions that comprise 1-6 carbon atoms (C₁-C₆ carboxylate) are illustrated by formate, acetate (CH₃CO₂⁻), propionate, butyrate, hexanoate, maleate, fumarate, oxalate, lactate, pyruvate, and the like, are also suitable for appropriate contemplated ionic liquid cations. Further examples include sulfonated or halogenated carboxylates like trifluoroacetate (TA; CF₃CO₂⁻).

Sulfate anions (SO₄⁻), such as tosylate, mesylate, trifluoromethanesulfonate or triflate (TfO; CF₃SO₂⁻), trifluoroethane sulfonate, di-trifluoromethanesulfonyl amino, nonaflate (NfO; CF₃(CF₂)₃SO₂⁻), bis(triflyl)amide (Tf₂N; (CF₃SO₂)₂N⁻), and heptaflurorobutanoate (HB; CF₃(CF₂)₃SO₂⁻), and xylenesulfonate (see WO2005017252, which is incorporated by reference herein for ionic liquids with anions derived from sulfonated aryls) are also suitable for use as the permanent anion for the disclosed cationic nucleoside analogs.

Still other examples of anions that can be present in the disclosed ILs include, but are not limited to, other sulfates, sulfites, bicarbonates, phosphates, phosphates, phosphites, nitrates (NO₃⁻), nitrites (NO₂⁻) and the like, including mixtures thereof.

Other suitable anions contemplated herein are docusate, saccharinate and acesulfamate. Saccharin, as an alkali metal salt, and acesulfame (6-methyl-3,4-dihydro-1,2,3-oxathiazin-4-one 2,2-dioxide), which has previously only been offered as potassium salt, are in widespread use in foodstuffs as non-nutritive sweeteners. Such anions can be used when one desires to prepare an ionic liquid composition that has sweetness as one of its desired properties. For example, saccharin and acesulfame can be combined with pharmaceutically active cations to prepare sweet tasting ionic liquids that have pharmaceutical activity.

Other examples of suitable anions that can be combined with the cationic nucleoside analogs disclosed herein include piperacillinate, penicillinate, folate, ibuprofenate, salicylate, acetylsalicylate, sulfacetamidate, naproxenate, benzoate, diclofenac, trans-cinnamate, and long chain polyunsaturated fatty acid carboxylate

### Methods of Making

The disclosed compositions can be prepared by combining one or more kinds of cations or cation precursors with one or more kinds of anions or anion precursors. This can be done to form an ionic liquid, which can be used as is or diluted by a solvent, or the ions or ion precursors can be mixed directly in a solution. Providing particular ions is largely based on identifying the desired properties of the ion (e.g., its charge and whether it has a particular bioactivity that is desired to be present in the resulting ionic liquid).

Further, when preparing a composition as disclosed herein, molecular asymmetry can be particularly desired. Low-symmetry cations and anions typically reduce packing efficiency in the crystalline state and lower melting points.

Once the desired ions are provided, the ions can be combined to form the disclosed ionic liquids. There are generally two methods for preparing an ionic liquid: (1) metathesis of a salt of the desired cation (e.g., a halide salt) with a salt of the desired anion (e.g., transition metal salt, like a Ag salt, Group I or II metal salt, or ammonium salt), such reactions can be performed with many different types of salts; and (2) an acid-base neutralization reaction. Another method for forming the disclosed ionic liquid compositions involves a reaction between a salt of a desired cation, say Cation X where X is an appropriate balancing anion (including, but not necessarily limited to, a halide), and an acid to yield the ionic liquid and HX byproduct. Conversely, the disclosed ionic liquid compositions can be formed by reacting a salt of a desired anion, say Y Anion where Y is an appropriate balancing cation, with a base to yield the ionic liquid and Y base byproduct.

For example, an anionic precursor can be treated with sodium or potassium hydroxide used in a molar ratio of from 0.7:3 to 0.8:5, in an aqueous environment at a temperature from 273 to 373K, e.g., 325K. The product, in the form of the sodium or potassium salt of the anion, can then undergo a reaction with the halide salt of a cation, as described herein, in the molar ratio of 1:0.7 to 1:1.5. Often during the reaction, the solvent can be completely evaporated to form the salt product.

The salts of the cations described herein and anions can also be prepared by an alternative procedure. A solution (preferably an aqueous or alcohol solution) of halide salts (e.g., chlorides, bromides or iodides) of the cations described herein can undergo anion exchange reactions with an anion exchange resin (preferably on an anion exchange column), to produce the cations with Off anions. Afterwards, a nucleoside (either in suspension or in solution) can be added to form the salts described herein, in a molar ratio from 1:0.7 to 1:1.5 at temperatures from 0 to 100° C. After the reaction, the solvent can be evaporated under reduced pressure and, after drying, new salts of the cations and the anions described herein can be isolated. For a review of the synthesis of ionic liquids see, for example, Welton, Chem Rev 1999, 99:2071-2083, which is incorporated by reference herein for at least its teachings of ionic liquid synthesis.

The purification of ionic liquids can be accomplished by techniques familiar to those skilled in the art of organic and inorganic synthesis, with the notable exception of purification by distillation of the ionic liquid. In some cases, ionic liquids can be purified by crystallization at appropriate conditions of temperature and pressure (e.g., at low temperature and pressure). Such techniques can include the use of a solvent from which the ionic liquid can be crystallized at an appropriate temperature.

### Methods of Use

The disclosed compositions have many uses. For example, the disclosed salts can be used to allow fine tuning and control of the rate of dissolution, solubility, and bioavailability, to allow control over physical properties, to improve homogenous dosing, and to allow easier formulations.

Converting an active nucleoside compound into an ionic liquid salt by introducing a permanent cation as a counter ion allows for enhancement of plant penetration and thus for improvement of delivery. These salts can increase the biological performance of the nucleoside due to the significantly increased water solubility as well as optional additional bioactivity introduced through the permanent cation. For example, permanent caions with recognized surface and transport properties can be paired with the nucleosides described herein resulting in intensified uptake and translocation of the active nucleoside compound.

The salts disclosed herein, which contain nucleosides, can be used in the same way as the nucleosides themselves in an in vivo setting. Thus, disclosed herein are methods of treating an individual infected or at risk of being infected with a virus with an effective amount of a nucleoside salt as disclosed herein, for example, a salt of acyclovir, as the anion, and a permanent cation such as choline, tributylmethylammonium, tetradecylmethylammonium, or other quaternary ammonium cation as discosed herein, or tetrabutylphosphonium.

The present disclosure is based upon the discovery that antiviral acyclovir or its prodrugs, can be deprotonated (converted into an anion) and paired with a permanent cation. Such salts of acyclovir showed improved solubility in aqueous media. The role of the permanent cation is to control the solubility and physical properties of the active salts, such as stability, hydrophobicity and melting point, etc. Permanent cations with a second biological functionality can be used to form dual active low-melting salts with acyclovir, the constituents of which, in combination, can achieve improved activity or synergistic effects. Examples include the use of antibacterial cations such as long-chain tetraalkylammonium compounds or vitamins such as choline.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Materials and Methods

All compounds (unless otherwise noted) were used as received without any further purification. Acyclovir and trimethylhexadecylammonium hydroxide 25% in methanol were purchased from TCI (Portland, Oreg.), choline hydroxide 45% in methanol was purchased from Aldrich (St. Louis, Mo.), and tetrabutylphosphonium hydroxide 40% (w/w) in water was bought from Alfa Aesar (Ward Hill, Mass.). Deionized water used in the solubility experiments and in obtaining the calibration curves was obtained with a specific resistivity of 17.25 MΩ⁻cm at 25° C. from a commercial deionizer by Culligan (Northbrook, Ill.).

All the ¹H, ¹³C, and ³¹P NMR spectra were recorded on a Bruker spectrometer (Madison, Wis.) 500 MHz Bruker Avance Spectrometer Bruker/Magnex UltraShield 500 MHz magnet (which was operating at 500 MHz for ¹H, 125 MHz for ¹³C spectra, and 202.5 MHz for ³¹P, respectively).

Infrared (IR) spectra were recorded on neat samples from 650-4000 cm⁻¹ using a Bruker α-FTIR on a diamond crystal.

The UV-VIS spectra used for development of calibration curves and for determination of the solubility values were performed on a Varian CARY 3 UV-Visible Spectrophotometer.

TGA experiments were performed on a Mettler Toledo TGA/DSC1 Star unit under a stream of nitrogen. Samples (5-20 mg) were placed on a platinum pan and were heated from 25° C. to 800° C. with a constant heating rate of 5° C./min and with a 30 min isotherm at 75° C. to remove any remaining volatiles. Decomposition temperatures (T_{5% onset}) were reported as the onset temperature with respect to the initial 5 wt % mass loss.

Melting points and phase transitions were measured on a Mettler Toledo DSC1 Star unit under a stream of nitrogen. Samples (5-20 mg) were placed in closed aluminum pan. The protocol used in the case of solid samples is as follows: (a) “ramp up” to the target temperature (T_{target}) at 5° C./min (where target temperature, T_{target}, is 50° C. below the measured T_{5% onset} obtained from TGA); (b) isotherm for 5 min; (c) “ramp down” at 5° C./min to about −50° C. (or colder if no crystallization is seen in the next heating scan) (d) “isotherm” for 5 min; (e) repeat steps (a)-(d) twice. For glasses the following protocol was used: (a) “ramp down” to about −100° C. at 5° C./min; (b) “isotherm” for 5 min; (c) “ramp up” at 5° C./min to about 100° C.; (d) “isotherm” for 5 min; (e) repeat steps (a)-(d) twice.

### Example 1

### Synthesis of Choline Acyclovir, [Cho][Acy] (1)

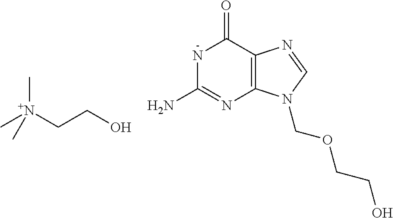

Acyclovir (0.693 mg, 3 mmol) was suspended in 20 ml of ethanol and a 46% solution of choline hydroxide in water (3 mmol) was added dropwise. The suspension was stirred for 15 min at room temperature until a clear solution was obtained and evaporated. Remaining volatile material was removed under reduced pressure (0.01 mbar, 50° C.) to yield choline acyclovir as yellow glass. ¹H-NMR (300 MHz, DMSO-d₆) δ (ppm)=7.4 (s, 1H), 5.2 (s, 2H), 4.9 (br s, 2H), 3.8 (s, 2H), 3.4 (m, 6H), 3.0 (s, 9H); ¹³C-NMR (125 MHz, DMSO-d₆) δ (ppm)=167.9, 161.8, 134.5, 118.9, 71.9, 70.4, 67.7, 60.3, 55.6, 53.5.

### Example 2

### Synthesis of tributylmethylammonium acyclovir, [N_{4,4,4,1}][Acy] (2)

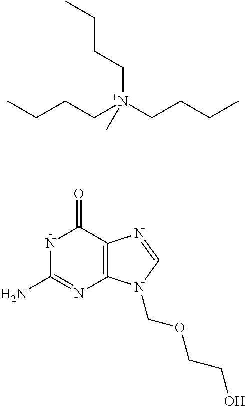 [SMILES: CCCC[N+](C)(CCCC)CCCC ; Nc1nc2c(ncn2COCCO)c(=O)[nH]1]

Prepared according to Example 1 to give tributylmethylammonium acyclovir as white solid. ¹H-NMR (500 MHz, d₆-DMSO) δ (ppm)=7.5 (s, 1H), 5.3 (s, 2H), 3.5 (s, 4H), 3.2 (m, 7H), 2.9 (s, 3H), 1.6 (m, 6H), 1.4 (m, 6H), 0.9 (m, 9H); ¹³C-NMR (125 MHz, DMSO-d₆) δ (ppm)=167.34; 161.54; 152.13; 134.47; 118.97; 71.98; 70.45; 60.86; 60.50; 48.01; 23.89; 19.65; 13.93.

### Example 3

### Synthesis of tetradecylmethylammonium acyclovir, [N_{1,1,1,16}][Acy] (3)

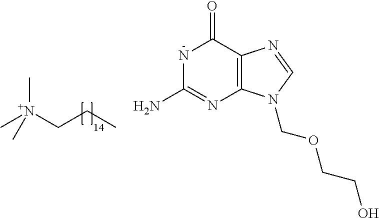

Prepared according to Example 1 to give trimethylhexadecylammonium acyclovir as white solid. ¹H-NMR (500 MHz, DMSO-d₆) δ (ppm)=7.49 (s, 1H), 5.8 (s, 1H), 5.29 (s, 2H), 3.46 (s, 4H), 3.27 (m, 3H), 3.05 (s, 9H), 1.64 (m, 2H), 1.24 (m, 26H), 0.85 (m, 3H); ¹³C-NMR (125 MHz, DMSO-d₆) δ (ppm)=166.31; 160.79; 152.29; 134.99; 118.53; 72.03; 70.49; 65.74; 60.51; 52.58; 31.75; 29.51; 29.47; 29.41; 29.27; 29.15; 28.97; 26.22; 22.54; 22.51; 14.39.

### Example 4

### Synthesis of tetrabutylphosphonium acyclovir, [P_{4,4,4,4}][Acy] (4)

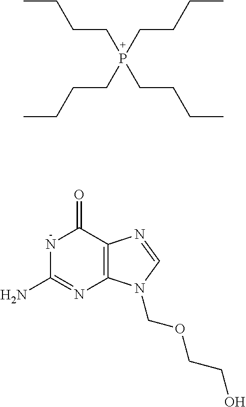

Prepared according to Example 1 to give tetrabutylphosphonium acyclovir as white solid. ¹H-NMR (500 MHz, DMSO-d₆) δ (ppm)=7.4 (s, 1H), 5.2 (s, 2H), 4.9 (br s, 1H), 3.4 (s, 4H), 2.2 (m, 8H), 1.4 (m, 16H), 0.8 (m, 12H); ¹³C-NMR (125 MHz, DMSO-d₆) δ (ppm)=167.72; 161.81; 151.97; 133.88; 119.22; 71.83; 70.29; 60.44; 23.71; 23.05; 17.71; 13.64; ³¹P-NMR (202.5 MHz, DMSO-d₆) δ (ppm)=34.07 (s).

### Example 5

### Synthesis of tributylmethylammonium acyclovir, [N_{4,4,4,4}][Acy] (5)

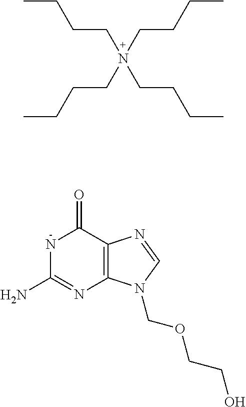

Prepared according to Example 1 to give tetrabutylammonium acyclovir as white solid. ¹H-NMR (500 MHz, DMSO-d₆) δ (ppm)=7.39 (s, 1H), 5.27 (s, 2H), 4.88 (br, 2H), 3.46 (s, 4H), 3.17 (m, 8H), 1.57 (m, 8H), 1.30 (m, 8H), 0.93 (m, 12H); DEPT 135 (125 MHz, DMSO-d₆) δ (ppm)=134.08 (CH, positive), 71.95 (CH₂, negative), 71.19 (CH₂, negative), 61.00 (CH₂, negative), 58.00 (CH₂, negative), 23.55 (CH₂, negative), 19.70 (CH₂, negative), 13.93 (CH₃, positive).

### Example 6

### Synthesis of acyclovir hydrochloride, [H₂Acy]Cl (6)

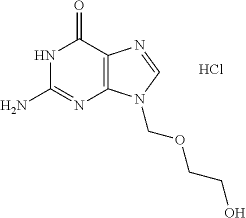 [SMILES: Nc1nc2c(ncn2COCCO)c(=O)[nH]1 ; Cl]

Acyclovir (3.00 g, 13.3 mmol) was suspended in 30 mL isopropanol. A solution of concentrated HCl (0.486 g, 13.3 mmol; 1.32 mL HC137% was used) in 15 mL isopropanol was added to this suspension and the reaction mixture was stirred for 30 min at room temperature. The solvent was evaporated using a Rotavapor and the solid obtained was further dried using high vacuum with no heating. The product was obtained as a white solid in a 94% yield. ¹H-NMR (500 MHz, DMSO-d₆) δ (ppm)=11.68 (s, 1H); 8.99 (s, 1H); 7.31 (s, 2H); 5.51 (s, 2H); 3.58 (t, 2H); 3.47 (t, 2H); ¹³C-NMR (125 MHz, DMSO-d₆) δ (ppm)=155.92; 154.56; 150.56; 138.02; 110.28; 74.13; 71.68; 60.30.

### Example 7

### Synthesis of acyclovir docusate, [H₂Acy][Doc] (7)

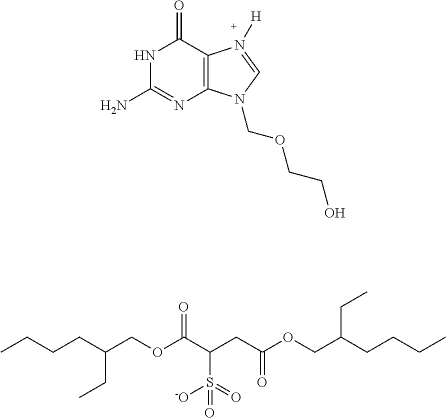

Acyclovir hydrochloride (1.5 g, 5.73 mmol) and silver docusate (3.03 g, 5.73 mmol) were suspended in 80 mL methanol and the resulting mixture was stirred for 10 h in dark and at room temperature. The suspension obtained was filtered through Celite and the resulting solution was evaporated using a Rotavapor at 40° C. The obtained residue was further dried under high vacuum and at 40° C. resulting in the formation of an off-white solid in 80% yield. ¹H-NMR (500 MHz, DMSO-d₆) δ (ppm)=11.30 (s, 1H); 8.97 (s, 1H); 7.01 (s, 2H); 5.49 (s, 2H); 3.89 (m, 4H); 3.65 (dd, 1H); 3.58 (m, 2H); 3.48 (m, 2H); 2.91 (dd, 1H); 2.81 (dd, 1H); 1.49 (m, 2H); 1.27 (m, 18H); 0.84 (m, 12H); ¹³C-NMR (125 MHz, DMSO-d₆) δ (ppm)=171.47; 168.78; 155.67; 154.68; 150.65; 138.13; 110.16; 74.22; 71.75; 66.68; 66.61; 66.57; 66.54; 61.92; 60.32; 38.65; 38.61; 38.57; 34.56; 30.21; 30.09; 30.03; 28.80; 23.67; 23.64; 23.49; 22.86; 22.83; 14.35; 14.32; 11.26; 11.23; 11.19.

### Example 8

### Solubility of Acyclovir ([HAcy]) in Deionized Water

20 mg Acyclovir free base was suspended in 10 mL de-ionized H₂O and stirred overnight at room temperature. The next day, there was still undissolved solid acyclovir. After filtering through Teflon syringe filters 0.2 μm), UV was used to determine the absorbance at 254 nm. A calibration curve of the compound in water was used to determine the concentration. The experiment was done in duplicate; solubility values were determined to be:

1^{st}: 1.422 g/L; 6.315×10⁻³ mol/L; 1.42 mg ‘acyclovir’/mL

2^{nd}: 1.418 g/L; 6.300×10⁻³ mol/L; 1.41 mg ‘acyclovir’/mL

Average: 1.42 g/L; 6.307×10⁻³ mol/L; 1.41 mg ‘acyclovir’/mL

Literature value: 1.3-1.6 mg/mL at 25° C. (5.77×10⁻³ mol/L).

### Example 9

### Solubility of [Cho][Acy] (1) in Deionized Water

In a vial, 5.500 g [Cho][Acy] was mixed with 2 mL DI H₂O. The mixture was stirred for 24 h at room temperature resulting in the formation of a very viscous solution. A certain amount of the mixture obtained (3.145 g) was transferred to another vial and centrifuged for 2 h 30 min but no phase separation was obtained. This mixture was allowed to stand for 15 days, at which point a thin layer of a more viscous mixture was seen at the bottom of the vial.

Two aliquots were taken from the top phase and each of them was filtered through Teflon syringe filter (0.2 μm) resulting in 2 viscous mixtures; 0.1 mL from each of these 2 mixtures were further diluted to 100 mL with DI H₂O and the absorbance measured using UV was out of the range of our spectrometer; therefore, 0.25 mL of this solution was diluted to 25 mL with DI H₂O and the absorbance at 254 nm of the obtained solution was measured.

A calibration curve of the compound in water was used to determine the concentration. Solubility values were determined to be:

1^{st}: 1038.30 g/L; 3.162 mol/L; 708.91 mg ‘acyclovir’/mL

2^{nd}: 1000.54 g/L; 3.047 mol/L; 683.14 mg ‘acyclovir’/mL

Average: 1019.42 g/L; 3.1045 mol/L; 696.03 mg ‘acyclovir’/mL.

### Example 10

### Solubility of [N_{1,1,1,16}][Acy] (3) in Deionized Water

In a vial, 0.500 g [N_{1,1,1,16}][Acy] was suspended in 1 mL DI H₂O. The mixture was stirred for 24 h at room temperature. The next day there was still undissolved solid [N_{1,1,1,16}][Acy]. After filtering through a Teflon syringe filter (0.2 μm), 0.1 mL solution was diluted to 25 mL with DI H₂O and its absorbance at 254 nm was determined.

A calibration curve of the compound in water was used to determine the concentration. The experiment was done in duplicate; solubility values were determined to be:

1^{st}: 250.81 g/L; 0.4930 mol/L; 110.53 mg ‘acyclovir’/mL

2^{nd}: 253.56 g/L; 0.4984 mol/L; 111.74 mg ‘acyclovir’/mL

Average: 252.18 g/L; 0.4957 mol/L; 111.13 mg ‘acyclovir’/mL

### Example 11

### Solubility of [P_{4,4,4,4}][Acy] (4) in Deionized Water

In a vial, 4.8 g [P_{4,4,4,4}][Acy] was suspended in 2 mL DI H₂O. The mixture was stirred for 24 h at room temperature. The next day a very viscous slurry suspension was obtained. After filtering through a Teflon syringe filter (0.2 μm), a clear viscous solution was obtained. 0.1 mL of the obtained solution was diluted to 100 mL with DI H₂O; the absorbance of this solution measured using UV was out of the range of the spectrometer; therefore, 0.5 mL of this solution was diluted to 25 mL with DI H₂O and the absorbance of the obtained solution was measured.

A calibration curve of the compound in water was used to determine the concentration. The experiment was done in duplicate; solubility values were determined to be:

1^{st}: 789.8 g/L; 1.6331 mol/L; 366.12 mg ‘acyclovir’/mL

2^{nd}: 817.99 g/L; 1.6913 mol/L; 379.20 mg ‘acyclovir’/mL

Average: 803.89 g/L; 1.6622 mol/L; 372.67 mg ‘acyclovir’/mL

### Example 12

### General Procedure for Determination of the Solubility by ¹H NMR

For all solvents used (water, Phosphate Buffer Solution (PBS), Simulated Gastric Fluid (SGF), Simulated Intestinal Fluid (SIF)) same amount of compound/solvent was used: 0.1 g compound/0.5 mL solvent. The solutions were stirred at room temperature for 24 h. The resulting suspensions were filtered through a 0.2 μm Teflon syringe filter to obtain clear solutions. Quantitative NMR was used to determine the final solubility value. Deuterated dmso (dmso-d₆) containing 0.05% (v/v) TMS (as internal standard) was used to run the ¹H-NMR. 0.1 mL of the filtered solutions were combined with 0.4 mL dmso-d₆ w/ 0.05% (v/v) TMS (corresponding to 1.836×10-6 mols TMS). The solubility values were determined from the molar ratio between the peaks corresponding to the compound and TMS.

### Example 13

### Solubility of [N_{4,4,4,4}][Acy] (5) in Deionized Water

Solubility was determined according to the procedure described in Example 12.

The solubility was determined to be: 444.7 g/L; 0.9528 mol/L; 213.66 mg ‘acyclovir’/mL

### Example 14

### Solubility of [H₂Acy]Cl (6) in Deionized Water

Based on its amphoteric character (acyclovir presents two pKₐ values, 2.27 and 9.25). So acyclovir can be deprotonated to form the corresponding acyclovir anion or, when in acidic medium, acyclovir can be protonated to form the corresponding acyclovir cation. Therefore, to explore the possibility of using an acyclovir cation with a permanent counter anion, the following experiments were conducted.

In a vial, 0.150 g [Acy]HCl was suspended in 1 mL DI H₂O. The mixture was stirred for 24 h at room temperature. The next day there was still undissolved solid [Acy]HCl. After filtering, 0.1 mL solution was diluted to 25 mL with DI H₂O and its absorbance was determined.

A calibration curve of the compound in water was used to determine the concentration. The experiment was done in duplicate; solubility values were determined to be:

1^{st}: 62.81 g/L; 0.240 mol/L; 54.05 mg ‘acyclovir’/mL

2^{nd}: 63.33 g/L; 0.242 mol/L; 54.66 mg ‘acyclovir’/mL

Average: 63.07 g/L; 0.241 mol/L; 54.27 mg ‘acyclovir’/mL

### Example 15

### Solubility of [H₂Acy][Doc] (7) in Deionized Water

In a vial, 0.2 g [Acy][Doc] was suspended in 1.5 mL DI H₂O. The mixture was stirred for 24 h at room temperature resulting in the formation of an emulsion. The obtained emulsion was stable for at least 48 h at room temperature. To about 0.3 mL of this emulsion, water was added dropwise (mixing well each time after water was added) until a more clear mixture was obtained. The same type of mixture was seen after standing at room temperature overnight. The mixture was sonicated for about 30 min; this process (add water, sonication) was repeated until a clear solution was obtained. 0.5 mL of the clear solution obtained was diluted to 50 mL with DI H₂O and UV was used to determine the absorbance at 254 nm.

A calibration curve of the compound in water was used to determine the concentration. The experiment was done in triplicate; solubility values were determined to be:

1^{st}: 8.46 g/L; 0.01306 mol/L; 2.95 mg ‘acyclovir’/mL

2^{nd}: 8.45 g/L; 0.01305 mol/L; 2.94 mg ‘acyclovir’/mL

3^{rd}: 8.46 g/L; 0.01306 mol/L; 2.95 mg ‘acyclovir’/mL

Average: 8.46 g/L; 0.01306 mol/L; 2.95 mg ‘acyclovir’/mL

### Spectroscopy Data

All of the synthesized compounds were characterized by NMR Spectroscopy, IR spectroscopy, and thermal analysis. IR Spectroscopy was used to show full ionization. In the case of compounds 1-4, the band from ˜1720 cm⁻¹ characteristic of the C═O group from acyclovir disappears and a new band appears at ˜1560 cm⁻¹, consistent with carboxylate formation which suggests full ionization; however, in the case of [Acy][Doc] (7), there is no significant difference in the shift for the C═O band from acyclovir cation when compared to the C═O band from acyclovir, suggesting that protonation takes place on the imidazole ring and does not influence this band too much. Also, the C═O band corresponding to the docusate anion and the C═O band from acyclovir cation are overlapping at ˜1720 cm⁻¹. The N—H bend (˜1610 cm⁻¹) from the acyclovir cation is shifted to higher wavelengths in [Acy][Doc] (7).

### Water Solubility Data

Solubility data obtained from Examples 8-15 above are summarized in Table 2 below. All the compounds had a higher water solubility than the acyclovir itself. The highest water solubility was obtained when the hydrophilic cation choline was used as permanent cation: 1019.42 mg [Cho][Acy] (1) could be dissolved in 1 mL water, corresponding to 696.06 mg of ‘acyclovir’ active being able to be ‘dissolved’ in water; this obtained value is almost 500 times higher than the water solubility of acyclovir (1.42 mg/mL). Improvement was also obtained when the less hydrophilic cations tetrabutylphosphonium ([P_{4,4,4,4}]), tetrabutylamonium ([N_{4,4,4,4}]) and trimethylhexadecylammonium ([N_{1,1,1,16}]) were used as permanent cations: [P_{4,4,4,4}][Acy] (4) showed a water solubility of 803.89 mg/mL, corresponding to 372.67 mg ‘acyclovir’ active per 1 mL water, [N_{4,4,4,4}][Acy] (5) showed a water solubility of 444.70 mg/mL, corresponding to 213.66 mg ‘acyclovir’ active per 1 mL water, while the [N_{1,1,1,16}][Acy] (3) water solubility was of 252.18 mg/mL, corresponding to 111.13 mg ‘acyclovir’ active per 1 mL water.

TABLE 2
Solubility in water.

| | | Water solubility | | |
|---|---|---|---|---|
| Compound | | | mg/mL | mg |
| | MW | mol/L | (g/L) | “acyclovir”/mL |
| Acyclovir* | 225.21 | 0.0063 (0) | 1.42 | 1.41 |
| [Cho][Acy] (1) | 328.37 | 3.1045 (81) | 1019.42 | 696.06 |
| [N_{1,1,1,16}][Acy] (3) | 508.74 | 0.4957 (4) | 252.18 | 111.13 |
| [P_{4,4,4,4}][Acy] (4) | 483.63 | 1.6622 (41) | 803.89 | 372.67 |
| [N_{4,4,4,4}][Acy] (5) | 466.66 | 0.9528 | 444.70 | 213.66 |
| [H₂Acy]Cl (6) | 261.71 | 0.2410 (1) | 63.07 | 54.27 |
| [H₂Acy][Doc] (7) | 646.77 | 0.0131 (0) | 8.47 | 2.95 |

*Literature values 1.3-1.6 mg/mL

Although of a smaller effect, [H₂Acy]HCl (6), and [H₂Acy][Doc] (7) also showed an improvement in the water solubility: [H₂Acy]HCl (6) showed a water solubility of 63.07 mg/mL (corresponding to 54.27 mg ‘acyclovir’/mL); while [H₂Acy][Doc] (7) exhibited a much lower water solubility of 8.48 mg/mL (corresponding to 2.95 mg ‘acyclovir’/mL) due to the hydrophobic character of the docusate anion, though that value is still two times higher than the water solubility of acyclovir. The lower water solubility values for compounds 6 and 7 derived from the acyclovir cation might be due to the formation of an imidazolium-type salt which leads to a decrease in the water solubility of the compounds.

### Simulated Biological Fluids Solubility Data

The solubility of acyclovir, [N_{4,4,4,4}][Acy] (5) and [H₂Acy]Cl (6), were also investigated in three different physiologically relevant aqueous environments: phosphate buffer saline (PBS, pH=7.4), simulated intestinal fluid (SIF, pH=6.8; without added pancreatin and prepared according to USP 26), and simulated gastric fluid (SGF, pH=1.2; without added pepsin and prepared according to USP 30). The solubility of neutral acyclovir was studied only in PBS and SIF, and the solubility of [H₂Acy]Cl (6) was investigated in SIF and SGF. Solubility experiments were conducted as described in example 12.

TABLE 3
Solubility in simulated biological fluids 9 mg ‘acyclovir’/mL)^{a}

| | PBS (pH = 7.4) | SIF (pH = 6.8) | SGF (pH = 1.2) |
|---|---|---|---|
| Acyclovir | 1.4^{b} | 2.22 | — |
| | 1.5 | | |
| [N_{4,4,4,4}][Acy] (5) | 1.0 | 144.0 | — |
| [H₂Acy]Cl (6) | — | 167.0 | 292.8 |

^{a}Determined using quantitative NMR;
^{b}Determined using UV-Vis

### TGA Data

Thermal analysis (thermogravimetric analysis, TGA, and differential scanning calorimetry, DSC) was used to characterize all the compounds obtained (Table 4). The isolated compounds varied from solid materials to glasses. DSC showed no melting point and a glass transition for ionic liquids [Cho][Acy] (1) and [N_{4,4,4,1}][Acy] (2), and [N_{4,4,4,4}][Acy] (5) ((1) with T_{g}=32° C., (2) with T_{g}=−15° C., and (5) with T_{g}=25° C.) and a melting point lower than body temperature for [N_{1,1,1,16}][Acy] (3) (Tₘ=12° C.); however, it was found that the phosphonium derivative [P_{4,4,4,4}][Acy] (4) has a melting point of Tₘ=126° C., higher than 100° C., a value that excludes this compound from being classified as an ionic liquid. The best thermal stability was obtained with a phosphonium-based cation: the salt [P_{4,4,4,4}][Acy] (4) showed a T_{5% onset} (onset for 5% decomposition from thermogravimetric analysis, TGA) of 221° C. higher than the ionic liquids 1-3 and 5.

TABLE 4
Thermogravimetric analysis and differential scanning
calorimetry results.

| Compound (#) | Appearance | Tₘ [° C.] | T_{5%onset} [° C.] |
|---|---|---|---|
| Acyclovir | White solid | 265 (dec); 245-246* | 240 |
| [Cho][Acy] (1) | Yellow glass | T_{g} = 32 | 128 |
| [N_{4,4,4,1}][Acy] (2) | Yellow solid | T_{g} = −15 | 115 |
| [N_{1,1,1,16}][Acy] (3) | White solid | 12 | 150 |
| [P_{4,4,4,4}][Acy] (4) | White solid | T_{g} = 8 | 221 |
| [N_{4,4,4,4}][Acy] (5) | White solid | T_{g} = 25 | 132 |
| [H₂Acy]Cl (6) | White solid | >209* | 156 |
| [H₂Acy][Doc] (7) | White solid | — | 122 |

*Determined using melting point apparatus

Since the bioavailability of the drugs is directly related to the dissolution rate of the drug, by improving the water solubility, the bioavailability of the compound should also be improved. Poorly soluble drugs (such as acyclovir) absorb slower than the more soluble ones, therefore research is focused on finding methods to increase the solubility of these drugs. The present disclosure shows that an ionic liquid strategy can be used to improve the solubility and therefore the bioavailability of acyclovir and other nucleoside compounds. By taking advantage of the amphoteric character of acyclovir, several ionic liquids and salts derived from acyclovir were successfully synthesized and their water solubility was determined to be much higher than that of acyclovir free base.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods and combinations of various features of the compounds and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

1. A salt, comprising: at least one kind of anion that is anion of a nucleoside analog and at least one kind of cation that is a permanent counter cation, or at least one kind of cation that is a cation of a nucleoside analog and at least one kind of anion that is a permanent counter anion, wherein the aqueous solubility of the salt is greater than the aqueous solubility of the nucleoside analog.

2. The salt of claim 1, wherein the nucleoside analog comprises an ionizable purine or pyrimidine base.

3. The salt of claim 1, wherein the nucleoside analog comprises a guanosine analog antiviral drug.

4. The salt of claim 3, wherein the guanosine analog antiviral drug comprises acyclovir or a pharmaceutically effective salt thereof.

5. The salt of claim 1, wherein the cation is an aprotic cation including quaternary nitrogen or a phosphorous or sulfur-containing analog thereof.

6. The salt of claim 1, wherein the cation is an ammonium cation of the structure NR¹R²R³R⁴, wherein R¹, R², R³, and R⁴ are each independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, or substituted or unsubstituted heteroaryl, or substituted or unsubstituted carbonyl.

7. The salt of claim 1, wherein the cation is a phosphonium cation of the structure ⁺PR¹R²R³R⁴, wherein R¹, R², R³, and R⁴ are each independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted heteroaryl, or substituted or unsubstituted carbonyl.

8. The salt of claim 1, wherein the cation is a sulfonium cation of the structure ⁺SR¹R²R³ wherein R¹, R², and R³ are each independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted heteroaryl, or substituted or unsubstituted carbonyl.

9. The salt of claim 1, wherein the cation is selected from the group consisting of N,N,N,N-tetraalkylammonium, N,N-dialkylpyrrolidinium, N-substituted pyridinium, N-substituted picolinium, N,N-disubstituted imidazolium, tetraalkylphosphonium, and trialkylsulfonium.

10. (canceled)

11. The salt of claim 1, wherein the cation is an antibacterial cation.

12. The salt of claim 1, wherein the cation is a long-chain tetraalkylammonium compound.

13. The salt of claim 1, wherein the anion of the nucleoside analog comprises anion of acyclovir and the cation is selected from a group consisting of choline, tetrabutylphosphonium, tributylmethylammonium, and trimethylhexadecylammonium.

14. The salt of claim 1, wherein the aqueous solubility of the salt is at least 100 times greater than the aqueous solubility of the nucleoside analog.

15. (canceled)

16. (canceled)

17. The salt of claim 1, wherein the salt is an ionic liquid at a temperature from about −30° C. to about 150° C.

18. The salt of claim 1, wherein the salt is an ionic liquid at a temperature from about 0° C. to about 120° C.

19. The salt of claim 1, wherein the salt is choline acyclovir.

20. The salt of claim 1, wherein the salt is tributylmethylammonium acyclovir or trimethylhexadecylammonium acyclovir.

21. The salt of claim 1, wherein the salt is tetrabutylphosphonium acyclovir.

22. The salt of claim 1, wherein the salt is acyclovir docusate or acyclovir chloride.

23. The salt of claim 1, wherein the anion is fluoride, chloride, bromide, iodide, C₁-C₆ carboxylate, trifluoroacetate, docusate, saccharinate, acesulfamate, piperacillinate, penicillinate, folate, ibuprofenate, salicylate, acetylsalicylate, sulfacetamidate, naproxenate, benzoate, diclofenac, trans-cinnamate, or long chain polyunsaturated fatty acid carboxylate.

24. (canceled)

25. A method of preventing and treating viral infection in an individual, the method comprising administering an effective amount of the salt of claim 1 to an individual.

26-35. (canceled)
